(19) United States
(12) Patent Application Publication
(10) Pub. No.: US 2011/0229516 A1
(43) Pub. Date: Sep. 22, 2011

(54) ADJUVANT PHASE INVERSION CONCENTRATED NANOEMULSION COMPOSITIONS

(75) Inventors: Maria G. Ochomogo, Pleasanton, CA (US); Ritu Saini, Pleasanton, CA (US); Richard J. Uriarte, Pleasanton, CA (US); Rachel Watson-Clark, Pleasanton, CA (US); Yuming Yang, Pleasanton, CA (US)

(73) Assignee: The Clorox Company, Oakland, CA (US)

(21) Appl. No.: 13/043,356

(22) Filed: Mar. 8, 2011

Related U.S. Application Data
(60) Provisional application No. 61/315,198, filed on Mar. 18, 2010.

(51) Int. Cl. A61K 39/12; A61K 39/02; A61K 39/002; A61P 37/00

(52) U.S. Cl. 424/204.1; 424/234.1; 424/265.1

(57) ABSTRACT

Vaccine adjuvant food nanoemulsion compositions comprising a food safe nonionic surfactant, a hydrophobic food flavorant (e.g., an essential oil), a kosmotrope, and water. The nanoemulsion composition can be used as a vaccine adjuvant composition to enhance inactivated antigens, including protein antigens. Such compositions may be delivered as a nasal or oral spray. The compositions may inherently contain natural antimicrobials and antioxidants and are advantageous over other compositions which often require preservatives, flavorings and antimicrobials.

### CROSS-REFERENCE TO RELATED APPLICATIONS

The present application claims the benefit of U.S. Provisional Patent Application Ser. No. 61/315,198 filed Mar. 18, 2010 and entitled NANOEMULSIONS COMPOSITIONS, which is hereby incorporated by reference in its entirety.

### BACKGROUND OF THE INVENTION

1. The Field of the Invention

Nanoemulsions used as mucosal vaccine adjuvants offer the possibility of achieving immunity with fewer antigens. Existing nanoemulsions are typically oil in water emulsions with average droplet sizes of about 400 nm. Such nanoemulsions should not be confused with nano particles. This small size allows the droplets to traverse mucosal membranes without disrupting normal tissues. Encapsulation of various targets such as antigens using nanoemulsions is an increasingly employed targeted delivery system for drugs or other medications. Of course, one readily recognizable benefit of a nanoemulsion delivery system is that it is needle free.

A classic oil-in-water microemulsion typically includes water, oil and one or more surfactants and co-surfactants. Typical compositions require at least two different surfactants. Although nanoemulsions can be formed kinetically, the selection of components, their relative amounts, and shear flow are critical factors in their formation. Such factors also affect final characteristics such as optical appearance, organoleptic characteristics (e.g., taste and smell), and thermodynamic stability. In addition, when such nanoemulsions are used as delivery systems in products, for example in nasal spray vaccines, they must fulfill all the requirements of such products. For example, such a product should provide shelf-life stability at typical storage temperatures of at least a few months without resulting in significant deterioration, rancid odor, or bitter flavor.

2. Description of Related Art

Emulsions have been widely used in vaccine and other pharmaceutical formulations for many years. Nevertheless, existing emulsions exhibit limited thermodynamic stability. For example, they separate into their two original liquid phases upon standing. Without being bound by theory, this is believed to occur because such compositions require kinetic energy to be input to the system in order to form the emulsions. Such a characteristic represents one of the greatest disadvantages of such compositions. Due to their limited stability over time, essentially all known emulsion based products undergo Ostwald ripening, cream formation, and finally phase separation.

Although the prior art may include microemulsions and nanoemulsions, they do not contain the same components and in the same amounts as the present invention, and more particularly, they do not combine them in a way to be thermodynamically stable, they do not extend the anti-oxidation properties of the vaccines, and/or they do not have an average droplet size of less than about 100 nm, more preferably less than about 50 nm on average. In addition, formulating aqueous adjuvant vaccine nanoemulsions with oil using a microfluidizer or a homogenizer can be challenging because: (1) they are not kinetically stable; (2) the shearing enhances oxidation of the oils; and (3) they are not readily miscible in water. As a result, essential oils and other oil based adjuvants are often difficult to prepare in a form that will allow them to be readily incorporated into an aqueous solution without the use of high shearing equipment and/or high temperatures.

There exists an unmet need for nanoemulsion formulations that are edible, substantially clear, thermodynamically stable, that are entirely composed of food grade quality components, which do not require at least two different surfactants, which comprise a relatively low level of surfactant in the patient delivered form (e.g., no more than about 15%, more preferably no more than about 10%) require no additional emulsifiers, and which provide excellent organoleptic qualities. In one embodiment of the invention, the adjuvant vaccines can be made using natural essential oils and/or distilled aroma oils derived from herbs, spices, fruits, plant components, or a blend of such components. Many of these inherently contain natural antimicrobial and/or antioxidative components dependent of their origin, resulting in an active with a pleasant flavor without the need for additional antimicrobials, antioxidants or preservatives to obtain a shelf stable product. In one embodiment, the composition does not contain the use of a vegetable oil (e.g. soy, peanut, canola oil, etc.) active component and a separate preservative and flavorant in the formulation. Furthermore, in one embodiment, the composition does not contain additional antioxidants since the surfactant encapsulates the natural essential oil(s) to prevent oxidation over time. Thus, the embodiment is a relatively simple composition capable of carrying antigens across the mucosal membrane. Furthermore, due to the large surface are per volume of the small nano-size droplets, there is an enhancement effect as to the availability of the antigen, resulting in enhanced vaccine efficiency and effectiveness.

### BRIEF SUMMARY OF THE INVENTION

In accordance with the above objects and those that will be mentioned and will become apparent below, one aspect of the present invention comprises an adjuvant vaccine emulsion composition comprising: (a) an antigen derived from bacteria, fungi, viruses, or parasites; (b) a food safe nonionic surfactant; (c) a hydrophobic food flavorant selected from the group consisting of: essential oil, oleoresin, oil-based natural flavors and combinations thereof; (d) a kosmotrope; and (e) water; (f) wherein the composition does not contain emulsifiers, phospholipids, triglycerides, vegetable oils, cholesterol, fatty acids, block polymers, trehalose dimycolate, cell wall skeleton, or mercury preservatives.

In accordance with the above objects and those that will be mentioned and will become apparent below, another aspect of the present invention comprises an adjuvant vaccine emulsion composition comprising: (a) an antigen derived from bacteria, viruses or parasites; (b) a food safe nonionic surfactant; (c) a hydrophobic natural flavorant selected from the group consisting of: essential oil, oleoresin, oil-based natural flavors and combinations thereof; (d) a kosmotrope selected from the group consisting of lactate, gluconate, sulfate, adipate, citrate, chloride, carbonate, bicarbonate, phosphate, pyrophosphate, nitrate, acetate and mixtures thereof; and (e) water; and (f) wherein the composition does not contain emulsifiers, phospholipids, triglycerides, vegetable oils, cholesterol, fatty acids, block polymers, trehalose dimycolate, cell wall skeleton, or mercury preservatives.

In accordance with the above objects and those that will be mentioned and will become apparent below, another aspect of the present invention comprises an adjuvant vaccine emulsion composition consisting essentially of: (a) an antigen derived from bacteria, viruses or parasites; (b) a food safe nonionic surfactant; (c) a hydrophobic natural flavorant selected from the group consisting of: essential oil, oleoresin, oil-based natural flavors and combinations thereof; (d) a kosmotrope; (e) water; and (f) optionally, osmotic agents, pressure regulators, glycerol, mannitol, preservatives, thiomersal, chlorobutanol, hypochlorous acid, parabens, antioxidants, alpha tocopherol, bioadhesive agents, natural gums, karaya, PGA, pectin, polyacrylic acid, polymethacrylates PAA copolymers, carbopol, carbomer, CMC, PVA and mixtures thereof; and (g) wherein the composition does not contain emulsifiers, phospholipids, triglycerides, vegetable oils, cholesterol, fatty acids, block polymers, trehalose dimycolate, cell wall skeleton, or mercury preservatives.

Further features and advantages of the present invention will become apparent to those of ordinary skill in the art in view of the detailed description of preferred embodiments below, when considered together with the attached claims.

### DETAILED DESCRIPTION OF THE PREFERRED EMBODIMENTS

### I. Definitions

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

References herein to “one embodiment”, “one aspect” or “one version” of the invention include one or more such embodiments, aspects or versions, unless the context clearly dictates otherwise.

As used in this specification and the appended claims, the singular forms “a,” “an” and “the” include plural referents unless the context clearly dictates otherwise.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In the application, effective amounts are generally those amounts listed as the ranges or levels of ingredients in the descriptions, which follow hereto. Unless otherwise stated, amounts listed in percentage (“%'s”) are in weight percent (based on 100% active) of the active composition alone, not accounting for the substrate weight. Each of the noted food emulsion composition components is discussed in detail below.

The precise definition of a “hydrophobic food flavorant” is difficult since its literal definition includes anything that contributes to vaccines like adjuvant, essential oils, antimicrobials, enzymes, flavorant etc. A legal definition by the U.S. Code of Federal Regulations of a natural flavorant is: “ the essential oil, oleoresin, essence or extractive, protein hydrolysate, distillate, or any product of roasting, heating or enzymolysis, which contains the flavoring constituents derived from a spice, fruit or fruit juice, edible yeast, herb, bark, bud, root, leaf or any other edible portions of a plant, meat, seafood, poultry, eggs, dairy products, or fermentation products thereof, whose primary function in food is flavoring rather than nutritional.” The term “hydrophobic food flavorant” includes such natural flavorants. The term “hydrophobic food flavorant” includes, but is not limited to essential oils, oleoresins, oil-based natural flavors, and mixtures or combinations thereof. An “essential oil” is any concentrated hydrophobic liquid containing volatile aroma compounds from plants. Essential oils are natural products commonly used in foods and beverages for their fragrance and taste properties. “Oleoresins” are a naturally occurring mixture of an oil and a resin extracted from various plants. Any other suitable oil-based natural flavors should also be included within the term “hydrophobic food flavorant”.

The term “food safe” refers to compositions, which are comprised entirely of materials that are considered food grade, and/or Generally Recognized As Safe (GRAS) and/or Everything Added to Food in the U.S. (EAFUS). In the United States, ingredients pre-approved for food use are listed in the United States Code of Federal Regulations (“C.F.R.”), Title 21. The term “food safe” includes compositions that are both safe and suitable for direct application to food work surfaces, including but not limited to, cutting boards, sinks, and kitchen counter tops, as well as direct food contact surfaces, including but not limited to, plates, platters and silverware. Food safe materials may also include ingredients that are well established as safe, or have adequate toxicological and safety pedigree, so that they can be added to existing lists or approved via a self-affirmation process.

The term “comprising”, which is synonymous with “including,” “containing,” or “characterized by,” is inclusive or open-ended and does not exclude additional, unrecited elements or method steps. See MPEP 2111.03. See, e.g., Mars Inc. v. H.J. Heinz Co., 377 F.3d 1369, 1376, 71 USPQ2d 1837, 1843 (Fed. Cir. 2004)

The term “consisting essentially of as used herein, limits the scope of a claim to the specified materials or steps “and those that do not materially affect the basic and novel characteristic(s)” of the claimed invention. In re Herz, 537 F.2d 549, 551-52, 190 USPQ 461, 463 (CCPA 1976). See MPEP 2111.03.

The term “consisting of as used herein, excludes any element, step, or ingredient not specified in the claim. In re Gray 53 F.2d 520, 11 USPQ 255 (CCPA 1931); Ex Parte Davis, 80 USPQ 448, 450 (Bd. App. 1948). See MPEP 2111.03.

### II. Introduction

In a broad sense, the present invention is directed to adjuvant emulsion compositions comprising a nonionic surfactant, a hydrophobic food flavorant, a kosmotrope, optionally a cationic polymer, and water. The adjuvant emulsions may be formulated so as to include an antigen (e.g., so as to form a vaccine) or may be delivered as an oral care oral rinse or oral spray. Such oral rinses or oral sprays may further include an additional antimicrobial component, although many of the preferred hydrophobic food flavorants (e.g., essential oils) will already serve this purpose. The composition generally is void of emulsifiers, phospholipids, triglycerides, vegetable oils, cholesterol, fatty acids, block polymers, trehalose dimycolate, cell wall skeleton, and mercury preservatives. The compositions may be initially formed as a concentrated water-in-oil microemulsion that upon dilution with water undergoes a phase inversion, resulting in a stable, substantially clear, oil-in-water nanoemulsion characterized by nano-sized hydrophobic droplets (e.g., average droplet size of less than 100 nm, preferably less than about 50 nm). The nanoemulsion remains stable even if heated or cooled (e.g., frozen or boiled) and can be stored for long periods of time (e.g., months or even years) without undergoing Ostwald ripening or phase separation into separate oil and aqueous phases. In addition, the hydrophobic food flavorant is encapsulated within the composition so as to prevent any malodor or bitter flavor resulting from oxidation of the hydrophobic food flavorant.

Unlike so called kinetically stable nanoemulsions, the PIC nanoemulsions, which may be referred to as micellar solutions, are transparent dispersions that form spontaneously without requiring any kinetic energy input. A concentrated microemulsion simply forms upon proper mixing of the components with each other. Upon addition of more water (i.e., dilution), the microemulsion phase inverts to form the desired stable nanoemulsion. Due to the very small size of dispersed oil-droplets (e.g., less than 100 nm in diameter), visible light is not scattered and therefore such microemulsions appear as clear or translucent and are significantly more thermodynamically stable than other emulsions. The small nano-size droplets are advantageously able to traverse the mucusal membrane. The compositions are made of food grade ingredients which are non irritating and exhibit no bitter taste. The compositions enhance the solubility of chemically unstable components through the use of the PIC nanoemulsion delivery system.

In the case of PIC nanoemulsions, these are stable upon infinite dilution so that the antigen may be combined with a previously prepared PIC nanoemulsion. In other words, one may add the antigen as part of the vaccine at one of various points. In addition, because the PIC nanoemulsions are stable upon infinite dilution, any desired amount of oil soluble and/or water soluble food grade components such as thickeners may be combined into a second water phase to adjust for viscosity (e.g., so that the vaccine will stay in your nose without dripping), thus eliminating the need for chemically derived adhesives. These characteristics result in significantly improved shelf-life stability (e.g., 3 years or more) over a large temperature range, and no formation of oxidation products that produce an offensive odor or bitter flavor. Another advantage of the PIC nanoemulsions composition is the high loads of actives that can be carried in the oil or water phase. Also, the PIC nanoemulsion's ingredients are less irritant than typical vaccine formulations, but less expensive and easier to manufacture (e.g., even as compared to a liposome delivery system).

Because the emulsion is stable, there is no need for, and the composition preferably is free of emulsifiers, phospholipids, triglycerides, vegetable oils, cholesterol, fatty acids, block polymers, trehalose dimycolate, cell wall skeleton, and mercury preservatives.

The nanoemulsion may initially be in the form of a concentrated water-in-oil microemulsion that becomes an oil-in-water nanoemulsion as it is diluted. Dilution triggers the inversion of the emulsion concentrate. The nanoemulsion may be substantially clear, spontaneously formed (i.e., without any kinetic energy input), and thermodynamically stable with an average droplet size of less than about 100 nm, more typically less than about 50 nm, and most preferably less than about 25 nm. This is a distinction from existing nanoemulsions that are opaque (e.g. milky appearance), formed kinetically (e.g., high shear), and which are kinetically stable but undergo Ostwald ripening and exhibit a relatively short shelf life.

Upon addition of the ingredients in the prescribed order, these formulations are spontaneously formed, are thermodynamically stable and are made as phase inversion concentrates. The nanoemulsions form upon mixing of an oil phase, an aqueous phase (i.e., including water), a nonionic surfactant, a kosmotrope, and optionally a cationic water soluble polymer. The nanoemulsions of the present invention form spontaneously and create a nano self-structured liquid (NSSL) that is thermodynamically stable over a wide range of temperatures and includes droplets in the size range of about 10 to about 100 nm, preferably from about 10 to about 50 nm and more preferably from about 5 to about 15 nm. The NSSL is also clear and stable upon dilution. The droplets size distribution depends on the amount and type of kosmotrope employed, type of cationic polymer employed (if any), and the ratio of kosmotrope to cationic polymer. Test results performed by the inventors show that this type of composition enhances flavor and antigen or protein activity, which characteristics enhance delivery of mucosal vaccine sprays. In one embodiment, the essential oil is encapsulated in the surfactant in one container and in another container, the kosmotrope is mixed with water. A concentrated emulsion is made by mixing ingredients in both containers, thus opening the one phase region in such a way that as one dilutes with water, the concentrated microemulsions go through a phase inversion that results that results in a translucent NSSL that is thermodynamically stable.

Some important characteristics, one or more of which may be present in any particular embodiment that allow their use as nasal sprays, oral rinses, or oral sprays include:

High payload. For example, up to 30% actives may be carried in the oil phase and/or water phase while maintaining Newtonian flow and low viscosity. Low payload limits an emulsion's ability to effectively serve as a nasal or oral spray.

Small droplet size. Typically less than about 50 nm, more preferably less than about 25 nm average (e.g., about 5 to about 15 nm). Once the nanoemulsion is formed through phase inversion of the concentrated microemulsion, the resulting nanoemulsion may be further diluted. Upon further dilution of the nanoemulsion, the droplet size remains substantially the same. This enhances the potency of actives, even at different doses. Also, it is instrumental in making these formulations since the enhancement is due to the high surface area to volume ratio resulting from nano-sized droplets and high energy curvature of the micelles.

Narrow droplet size distribution. This allows delivery of reproducible doses and ability to manipulate droplet size. For example, preferably at least about 50%, more preferably at least about 75% of the droplets lie within ±10 nm of the average value.

Made with food grade components, many of which are natural.

Substantially clear or translucent and thermodynamically stable upon dilution for an indefinite period of time. Because the kosmotrope and cationic polymer break the lamellar phase and open the one phase region, one can make these emulsions as a concentrated microemulsion. Addition of water spontaneously results in a phase inversion, resulting in the desired nanoemulsion, which can be further diluted without risk of phase separation. Because of their thermodynamic stability, the emulsions do not phase separate, even after long storage (e.g., months, or even years). Because of their thermodynamic stability, the emulsion remains stable even upon shearing as a result of dispensing the composition through a spray pump nozzle. Similarly, high electrolyte loading is possible.

Optional rheology modifiers. Such thickeners may be added as needed for retention time on the mucosa.

Optional ingredients. Although not needed, the composition may include other ingredients such as aluminum salts, egg protein, or preservatives typically used in vaccines. Of course, the above listing is not exhaustive, and various other ingredients may also be included.

Enhancement of flavors, antigens, and any antimicrobial agents as a result of the small droplet size.

Use of essential oils. The use of essential oils provides the emulsion with natural antimicrobial and antioxidant characteristics. The addition of other antimicrobial agents is not required (but is optional).

Protection from oxidation. Because the essential oil or other hydrophobic flavorant is encapsulated by the surfactant, it is protected indefinitely from oxidation.

Very low contact angle for superior wetness and spreadability in the oral cavity.

Drying studies have shown that the droplets dry uniformly and Fourier Transform Infrared Spectroscopy (FTIR) mapping shows that the droplets dry while their ingredients remain intact (i.e., encapsulation is maintained). This is not typical of other nanoemulsions. In addition, dried nanoemulsions can be rehydrated, so that upon contact with moisture the actives are released. This allows a dried nanoemulsion to be stored until activated by moisture. Repeated drying and rehydrating is also possible.

### III. Actives

Actives components include any antigens (including proteins) derived from bacteria, fungi, viruses, or parasites. The antigen may be water soluble, lipid soluble, or amphiphilic. The antigen may be added intrinsically, during preparation of the nanoemulsion, or extrinsically, added after the nanoemulsion has already been prepared. The nanoemulsion composition may be formulated as either a concentrated or dilute composition which can be used as a nasal spray delivered vaccine, or as an oral care spray. As a nasal spray vaccine, the antigen and flavorant components are delivered with enhanced effectiveness. As an oral care spray, the active component may include an antimicrobial agent, which may simply be the hydrophobic flavorant (e.g., in the case where the hydrophobic flavorant exhibits antimicrobial properties). Of course, the hydrophobic flavorant included in the nasal spray vaccine may also be considered an active component. In each embodiment, the active components exhibit enhanced effect. Concentrated nanoemulsion compositions can easily be further diluted to reduce the dosage of actives delivered, as needed.

The concentration of the antigen may depend on its nature. In the case of a vaccine, the antigen, which may include proteins, may be included in an amount of at least about 0.00001%. For example, typical amounts may range between about 0.00001% and about 10%, more typically between about 0.00001% and about 1% by weight of the composition.

Examples of peptide antigens may generally include, but are not limited to, natural and synthetic glycoprotein, covalently conjugated lipopeptides, protein toxoids (chemically or physically inactivated), non toxic bacterial surface structures or outer membrane proteins, natural or synthetic peptides and proteins derived from bacteria, viruses, fungi, or parasites.

More specific examples of antigens may include, but are not limited to, gp160, the envelope protein of HIV or a part thereof, Staphylococcus Enterotoxin B (SEB) toxoids (e.g., SEB toxoid C or F, any of which may be carboxymethylated, and/or physically or chemically inactivated, as desired), Lipopolysaccharide (LPS) antigen from S. flex, gp63, a surface protein antigen of Leishmania major (e.g., lipopeptides obtained from the major glycoprotein of the Leishmania parasite), natural or synthetic glycoprotein derived from dipsomania strains, non-toxic bacterial surface structures of Escherichia coli strains such as the Shiga-like toxin B Subunit (SLT-B) or AF-RL, Hepatitis B surface antigen, malaria antigen derivatives from different portions of Plasmodium faciparum, and influenza antigen derived from influenza viruses. Antigens may be prepared as will be known to those of skill in the art. For example, the antigen may be complexed (e.g., non-covalently) to a proteosome (e.g., meningococcal outer membrane proteins purified from Neisseria meningitides) to enhance the immunogenicity of the antigen. Similarly, glycopeptides or a part thereof may be conjugated (e.g., covalently attached) to a hydrophobic foot such as lauryl cysteine. Other antigens, as well as specific methods of preparation that may be employed will be apparent to one of skill in the art in light of the present disclosure.

In another embodiment, the active may be an antimicrobial agent. Examples of an antimicrobial agent includes but is not limited to, cetylpyridinum chloride (cross-linked to cationic polymer or stand alone), and organic acids (i.e. benzoic acid or sorbic acid). In another embodiment, the antimicrobial agent may be the hydrophobic flavorant.

### IV. Flavorant Actives

The hydrophobic flavorant active components may be present at levels from about 0.001% to about 15% by weight of the emulsion composition, preferably from about 0.01% to 10%, and most preferably from about 0.1% to about 5%. In one embodiment, the flavorant may comprise between about 0.5% and about 5% of the composition. The hydrophobic flavorant may provide several benefits attributable at least in part to this single component. For example, the flavorant provides a flavor and pleasant scent to the emulsion, and because many natural hydrophobic food flavorants include antioxidants and/or exhibit antimicrobial properties, separate components selected to provide these benefits are not required. In other words, the emulsion can exhibit antimicrobial and antioxidative properties without requiring separate components tailored to this purpose.

Any suitable essential oil, oleoresin, or oil-based natural flavorant may be used as the hydrophobic food flavorant. Essential oils preferred for use in the compositions are those essential oils which can form a microemulsion concentrate when combined with a water carrier and a nonionic surfactant. As explained above, the microemulsion concentrate is phase inverted upon addition of more water, resulting in the desired nanoemulsion. Suitable essential oils include, but are not limited to, those obtained from mint, tea tree, oregano, parsley, thyme, cloves, lemongrass, lemons, limes, grapefruit, oranges, anise, clove, roses, lavender, citronella, camphor, sandalwood, cedar, pine, rosemary, cynamic aldahyde, cinnamon, eucalyptus, peppermint, marjoram, garlic, onion, roasted spices, and combinations thereof. Preferred essential oils include mint oil, tea tree oil, lavender oil, pine oil, rosemary oil including Herbalox from Kalsec (unflavored), parsley oil, orange oil, onion oleoresins, lemongrass oil, lemon oil, Thyme oil, orange oil, clove oil, and combinations thereof. The most preferred essential oils include mint oil, tea tree oil, rosemary (flavored or unflavored), lemon oil, orange oil, lime oil, grapefruit oil, mint oil, parsley oil, lemongrass oil, and combinations thereof.

Other suitable essential oils include, but are not limited to, Anethole 20/21 natural, Aniseed oil china star, Aniseed oil globe brand, Balsam (Peru), Basil oil (India), Black pepper oil, Black pepper oleoresin 40/20, Bois de Rose (Brazil) FOB, Borneol Flakes (China), Camphor oil, White, Camphor powder synthetic technical, Cananga oil (Java), Cardamom oil, Cassia oil (China), Cedarwood oil (China) BP, Cinnamon bark oil, Cinnamon leaf oil, Citronella oil, Clove bud oil, Clove leaf, Coriander (Russia), Coumarin 69° C. (China), Cyclamen Aldehyde, Diphenyl oxide, Ethyl vanillin, Eucalyptol, Eucalyptus oil, Eucalyptus citriodora, Fennel oil, Geranium oil, Ginger oil, Ginger oleoresin (India), White grapefruit oil, Guaiacwood oil, Gurjun balsam, Guava, Passion Fruit, Coconut, Heliotropin, Isobornyl acetate, Isolongifolene, Juniper berry oil, L-methyl acetate, Lavender oil, Coriander oil, Lemon oil, Lemongrass oil, Lime oil distilled, Litsea Cubeba oil, Longifolene, Menthol crystals, Methyl cedryl ketone, Methyl chavicol, Methyl salicylate, Musk ambrette, Musk ketone, Musk xylol, Nutmeg oil, Orange oil, Patchouli oil, Peppermint oil, Phenyl ethyl alcohol, Pimento berry oil, Pimento leaf oil, Rosalin, Sandalwood oil, Sandenol, Sage oil, Clary sage, Sassafras oil, Spearmint oil, Spike lavender, Spike tea oil, Tagetes, Vanillin, Vetyver oil (Java), Wintergreen, Allocimene, ARBANEX, ARBANOL, Bergamot oils, Camphene, Alpha-Campholenic aldehyde, I-Carvone, Cineoles, Citral, Citronellol Terpenes, Alpha-Citronellol, Citronellyl Acetate, Citronellyl Nitrile, Para-Cymene, Dihydroanethole, Dihydrocarveol, d-Dihydrocarvone, Dihydrolinalool, Dihydromyrcene, Dihydromyrcenol, Dihydromyrcenyl Acetate, Dihydroterpineol, Dimethyloctanal, Dimethyloctanol, Dimethyloctanyl Acetate, Estragole, Ethyl-2 Methylbutyrate, Fenchol, FERNLOL, FLORILYS, Geraniol, Geranyl Acetate, Geranyl Nitrile, GLIDMINT Mint oils, GLIDOX, Grapefruit oils, trans-2-Hexenal, trans-2-Hexenol, cis-3-Hexenyl Isovalerate, cis-3-Hexanyl-2-methylbutyrate, Hexyl Isovalerate, Hexyl-2-methylbutyrate, Hydroxycitronellal, lonone, Isobornyl Methylether, Linalool, Linalool Oxide, Linalyl Acetate, Menthane Hydroperoxide, I-Methyl Acetate, Methyl Hexyl Ether, Methyl-2-methylbutyrate, 2-Methylbutyl Isovalerate, Myrcene, Nerol, Neryl Acetate, 3-Octanol, 3-Octyl Acetate, Phenyl Ethyl-2-methylbutyrate, Petitgrain oil, cis-Pinane, Pinane Hydroperoxide, Pinanol, Pine Ester, Pine Needle oils, Spice oils, alpha-Pinene, beta-Pinene, alpha-Pinene Oxide, Plinol, Plinyl Acetate, Pseudo lonone, Rhodinol, Rhodinyl Acetate, alpha-Terpinene, gamma-Terpinene, Terpinene-4-OL, Terpineol, Terpinolene, Terpinyl Acetate, Tetrahydrolinalool, Tetrahydrolinalyl Acetate, Tetrahydromyrcenol, TETRALOL, Tomato oils, Vitalizair, ZESTORAL, and combinations thereof.

The flavorant may include an oleoresin. Suitable types of oleoresins, include but are not limited to, basil, black pepper, capsicum, cardamom, celery seed, clove, garlic, coriander, cumin, fennel, fenu Greek, ginger, garlic, chili, green pepper, onion, nutmeg, anise, tumeric, vanilla, paprika, and any other oleoresin suitable for use in food emulsions.

In addition or alternative to essential oils and oleoresins, the flavorant may comprise other natural oil-based flavorants (e.g., plant isoflavons, hydrophobic aromas, etc.). The flavorant in the emulsion composition may include any mixtures or combinations of essential oils, oleoresins, natural oil-based flavorants, or combinations thereof.

### V. Non-Ionic Surfactants

The concentration of one or more nonionic surfactants included within compositions may depend on the amount of hydrophobic food flavorant active used, as well as on the levels deemed acceptable as food safe. In one embodiment, the nonionic surfactants are present at a concentration of at least about 0.2% by weight of the composition, preferably from about 1% to about 60%, more preferably from about 3% to about 15% and most preferably from about 5% to about 10%. In the case of a concentrate, the nonionic surfactant may comprise as much as about 60% of the composition. Such compositions will typically be diluted prior to use to the levels indicated above (e.g., no more than 15%, no more than about 10%). In one embodiment of the invention, the ratio of hydrophobic flavorant to nonionic surfactant is between about 1:2 and about 1:12, more preferably between about 1:2 and about 1:10, and most preferably between about 1:2 and about 1:5. The one or more nonionic surfactants selected preferably have an HLB of about 10 or greater, more preferably about 12 or greater. Higher HLB values correspond to increased hydrophobicity of the surfactant. The HLB value of a nonionic surfactant blend is determined by reference to the weighted average of the blended surfactants. An HLB value of 10 or greater aids in the formation of a translucent or clear nanoemulsion.

In one embodiment of the invention, suitable nonionic surfactants have a pour point of about 20° F. and are viscous but pourable within a temperature range of about 34° F. to about 40° F. Examples of suitable food safe nonionic surfactants include, but are not limited to, glycosides, sorbitan esters, ethoxylated sorbitan esters, polysorbates, polysorbate esters (e.g., TWEEN 20, 60, 80, or 85 available from Croda), sorbitan tristreate, monoglycerides, sucrose esters (e.g., available from Croda), ethoxylated castor oils, polyglycerol esters (Stepan's Drewpol 10-1-cc), and combinations thereof.

Optional additional water dispersible or at least partially water-soluble surfactants that may be included in some embodiments include alkylene oxide adducts of either fatty acids or partial fatty acid esters, for example, ethoxylated fatty acid partial esters of such polyols as anhydrosorbitols, glycerol, polyglycerol, pentaerythritol, and glucosides, as well as ethoxylated monodiglycerides, sorbitan trioleate, and aliphatic polyoxyethylene ethers such as polyoxyethylene (23) lauryl ether. However, as described above, in one embodiment, the composition does not contain phospholipids (e.g., lecithin), triglycerides, or fatty acids.

Preferred surfactants include sorbitan esters, for example polyoxyethylene sorbitan fatty acid esters or mixtures thereof such as those sold under the trademark TWEEN 20 (polyoxyethylene (20) sorbitan monolaurate), TWEEN 60 (polyoxyethylene (20) sorbitan monostearate), TWEEN 80 (polyoxyethylene (20) sorbitan monooleate) (each commercially available from ICI Americas Inc. of Wilmington, Del.), ethoxylated monodiglycerides or mixtures thereof such as those sold under the name Mazol 80 MGK (commercially available from Mazer Chemical, Inc. of Gurnee, Ill.), sorbitan trioleate (commercially available from ICI Americas Inc. under the name Span 85).

Other suitable food safe nonionic surfactants may comprise a glycoside surfactant. In one embodiment the glycoside surfactant is an alkyl polyglycoside. The alkyl polyglycoside surfactant may have linear or branched alkyl groups. Suitable alkyl polyglycoside surfactants preferably include a linear alkyl group. In preferred embodiment of the invention, the alkyl polyglycoside has between about 6 and about 22 carbons, more preferably between about 6 and about 12 carbons, even more preferably between about 6 and about 10 carbons and most preferably between about 8 and about 10 carbons.

### VI. Kosmotropes

Kosmotropes, which contribute to the stability and structure of water-water interactions, may act as emulsion stabilizers, promote uniformity of flavor transfer, and act as a release agent where it is desirable for the composition or device incorporating the composition to be peelable. Food grade hydroxylated kosmotropes break the lamellar liquid crystalline phase and facilitate the formation of nano-sized droplets with high curvature that leads to a pronounced change in the free energy, resulting in thermodynamic stability. Examples of kosmotropes suitable for use include, but are not limited to, glycerin, sugar alcohols (e.g., sorbitol), branched polyols (e.g., propylene glycol), and salts. More specific examples of sugar alcohols include, but are not limited to, sorbitol, propanediol, xylitol, arabitol, lactitol, maltitol, glycerol, mannitol, isomalt, erythritol, and mixtures thereof. More specific examples of suitable salts include, but are not limited to, lactate, gluconate, sulfate, adipate, citrate, chloride, carbonate, bicarbonate, phosphate, pyrophosphate, nitrate, acetate and mixtures thereof. Lactate and particularly gluconate are preferred kosmotrope salts.

Kosmotropes may be present in an amount of at least about 0.01%, more preferably from about 0.01 to about 20%, more preferably about 0.1% to about 10%, and most preferably about 0.5% to about 5% by weight of the composition. In one embodiment, the composition includes at least as much kosmotrope as the hydrophobic flavorant (i.e., at least about a 1:1 ratio). The ratio of hydrophobic essential oil to kosmotrope may be between about 1:1 and about 1:2.

### VII. Cationic Polymers

In preferred embodiments, the emulsion composition further includes a cationic water soluble polymer which acts in conjunction with the kosmotrope to stabilize the composition. Preferably, the concentration of polymer is approximately equal to the concentration of the kosmotrope (e.g., about a 1:1 ratio). The cationic polymer may comprise at least about 0.2% of the composition by weight. In one embodiment, the composition includes at least as much kosmotrope as the essential oil (i.e., at least about a 1:1 ratio), and there is at least about twice as much food safe nonionic surfactant as the hydrophobic flavorant (i.e., at least about a 2:1 ratio), at least about 0.2% cationic polymer, with the remainder being water. In this embodiment the ratio of hydrophobic essential oil to kosmotrope may be between about 1:1 and about 1:2.

Exemplary polymers include galactomannans, which are polysaccharides including a mannose backbone with galactose side group(s). According to one embodiment, the cationic polymer may have a relatively high charge density of at least about 0.05 milliequivalents per gram (meq/g).

Specific examples of suitable polymers include, but are not limited to, carrageen, carrageenan, guar gum, cassia gum, and chitosan. More specific suitable examples include cassia hydroxypropytriamonium chloride (1.9-3 meq/g), cat guar (e.g., JAGUAR from Rodhia and COMITIA from Cognis (0.98 meq/g)), cat carrageen (SAKEM type J) from FMC, carrageenan cetylpiridium chloride, and cross linked carrageen+Zn lactate.

In one embodiment, the polymer comprises a bilayer in which the first layer is an anionic polymer and the second layer is the cationic polymer. Such a bilayer is advantageous as it protects the antigen within the encapsulated droplet from environmental conditions, and can provide a targeted delivery system. For example, such a bilayer can protect the antigen from the low pH of the stomach in the case of oral administration of the vaccine. Such a bilayer may also provide targeted delivery when the pH meets a minimum threshold (e.g., about 7 or higher) so as to target release into the mucus membrane or intestines. Such an embodiment may provide for delivery of different antigens at different times or different places.

Such a polymer bilayer may be formed by any technique that will be apparent to those of skill in the art. For example, the emulsion concentrate including the hydrophobic flavorant (e.g., an essential oil), an aqueous phase, the nonionic surfactant, and a kosmotrope may be prepared. Within a separate container, the dilution portion that will be used to dilute the emulsion may be prepared. To the dilution water portion is added a bioactive to be entrapped and a solution of calcium chloride or other suitable salt. This is mixed under high shear (e.g., about 8,000 rpm or greater). A buffered solution of a suitable acid (e.g., alginic acid) is added and crosslinked with the calcium chloride salt to form anionic charged particles. Typical ratios of calcium chloride salt to alginic acid are about 1:6. Next, a suitable cationic polymer (e.g., chitosan, cationic carrageen, or other food grade starch) is added to the mixture. Typical ratios of anionic polymer (the alginate) to cationic polymer may be between about 1:1 and about 1:2, more preferably between about 1:1 and about 1:1.5. The nanoparticles solution is then mixed with the dilution water portion including the polymers. The cationic polymer attaches around the anionic polymer, forming a bilayer encapsulating the bioactives that will be released when the pH is appropriate (e.g., greater than about 7). In other words, the polymer bilayer protects the encapsulated bioactive until it encounters a pH greater than 7 so that the bioactive is protected within the stomach, and released in the intestines (or other appropriate location).

The diameters of the droplets will depend on the amount and indentity of polymers, and the diameter can be measured by measuring the total charge. For example, cationic polymer coated droplets preferably have a zeta potential from about 1 mV to about 60 mV, and the droplets have an average diameter of less than 100 nm, preferably less than about 50 nm, more preferably less than 25 nm (e.g., between about 5 nm and about 15 nm). Droplet size and distribution may be measured using dynamic light scattering (DLS).

### VIII. Water and Co-Solvents

In one embodiment, the emulsion composition may include other solvents in addition to water. Suitable other solvents include, but are not limited to, food grade C₂ to C₈ alcohols, including but not limited to, ethanol, glycerol, sugar alcohols or mixtures and/or isomers thereof. In one embodiment, the food grade emulsion composition is free of monohydric alcohols, including but not limited to ethanol, methanol, isopropanol, n-propanol, and t-butanol. In another embodiment, the food grade emulsion contains a single alcohol co-solvent without any additional polyol solvents. Other non-limiting examples of suitable solvents include: glycerin, 1,3-propanediol, propylene glycol, and mixtures thereof. When present, the other solvents typically comprise between about 8% and about 25% by weight of the composition, preferably between about 10% and about 20%, and most preferably between about 10% and about 15% by weight of the composition.

The amount of water in the concentrated nanoemulsion may be greater than 0% by weight, more preferably greater than 10% by weight and most preferably greater than 45% by weight. The majority of the food grade emulsion composition typically comprises water once it is diluted.

### IX. Buffers

Optionally, buffering and pH adjusting agents can be added to the food grade emulsion composition. Suitable buffers include, but are not limited to, organic acids, mineral acids, alkali metal and alkaline earth salts of silicate, metasilicate, polysilicate, carbonate, phosphate, polyphosphate, pyrophosphates, triphosphates, and tetraphosphates. Additional buffering agents may include nitrogen-containing materials. Examples of nitrogen-containing buffering agents include amino acids (e.g., lysine), MSG, disodium glutamate, and combinations thereof. Other suitable buffers include potassium citrate, ammonium carbamate, citric acid, and acetic acid. Combinations of any of the above buffering agents may also be acceptable. In one embodiment the composition contains only citric acid or citrate buffers (e.g., sodium citrate) and is free from any other types of buffers. For additional buffers see McCutcheon's Emulsifiers and Detergents, North American Edition, 1997, McCutcheon Division, MC Publishing Company Kirk and WO 95/07971, both of which are incorporated herein by reference.

When employed, the buffering agent may typically comprise at least about 0.001%, preferably between about 0.01% and about 10%, and more preferably between about 0.05% and about 2%.

### X. Additional Ingredients

In another embodiment, the composition optionally includes one or more incidental ingredients. The adjuncts include, but are not limited to dyes and/or colorants, vitamins, minerals, solubilizing materials, rheology modifiers (i.e., thickeners), foam controlling agents, hydrotropes, enzymes, radical scavengers, antioxidants, stabilizers, osmotic agents, pressure regulators, preservatives, and cloud point modifiers, chlorobutanol, hypochlorous acid, parabens, alpha tocopherol, bioadhesive agents (e.g., chitosan), natural gums, karaya, PGA, pectin, polyacrylic acid, polymethacrylates, PAA copolymers, carbopol, carbomer, CMC, and PVA. It is preferred that the compositions do not include mercury preservatives like thiomersal.

Suitable rheology modifiers can be obtained from Sigma Chemicals Co., located in St Louis, Mo. Exemplary rheology modifiers may include a water soluble polymeric shear thinning thickener such as Xanthan gum (e.g., KELTROL from Kelco). Such components may typically be included (if present) between about 0.001% and about 5% by weight of the composition.

Upon addition of the oil actives, nonionic surfactant, kosmotropes, polymers and water in the prescribed order, a thermodynamically stable phase inversion nanoemulsion is spontaneously formed. The nanoemulsions form spontaneously and create a NSSL that is thermodynamically stable over a wide range of temperatures and includes, on average, droplets in the size range of about 10 to about 100 nm, preferably from about 10 to about 50 nm and more preferably less than about 25 nm, for example from about 5 to about 15 nm. The NSSL is substantially clear and maintains these characteristics upon further dilution. These qualities allow the food grade emulsion composition to be formulated either as a concentrate that could be used to add to a vaccine antigen in the form of an adjuvant emulsion prior to use or as a more dilute nasal spray product where the active antigen is provided in a ready to use product. In one embodiment, a concentrated form could be diluted with water and an antigen (for vaccine product) or an antimicrobial (for an oral rinse product) to make vaccines, nasal sprays and oral rinse or spray products that very effectively spread and cover the oral or nasal cavity due to their low contact angle.

The nano-sized droplets or micelles are less than 100 nm, more preferably less than 50 nm in size, the dispersed system exhibits thermodynamic stability and behaves like a much larger, swollen micelle. Other adjuvant nanoemulsions require the addition of co-emulsifiers (i.e., a second emulsifier) with similar chain length and high shear (a microfluidizer), so the relatively large radius with almost parallel packing of emulsifiers will be optimal. In contrast, the present inventive nanoemulsions this case, do not require a second emulsifier or surfactant or microfluidizer to form thermodynamically stable micelles having a droplet size of less than 100 nm. Rather, the micelles are spontaneously formed due to the ingredients employed, and the order of addition.

The distribution of droplet sizes is relatively narrow. For example, for an average droplet size of 25 nm, at least about 70% of the droplets may lie within ±4 nm of this average value. More generally, at least about 50%, and more preferably at least about 75% of the droplets lie within ±10 nm of the average value.

Furthermore, the NSSL concentrate may be diluted as desired in either oil or water while maintaining the single phase emulsion including nano-sized droplets. The NSSL concentrates form a clear and transparent liquid that produces no precipitates, crystalline matter or turbidity. The structured concentrate is of relatively low viscosity (e.g., less than about 30 centipoise), is thermodynamically stable, does not separate, coalesce, aggregate, flocculate or cream at storage temperatures, even after prolonged storage.

The nanoemulsion compositions can be packaged within any suitable container known to one skilled in the art. It may be packaged as a concentrate in suitable containers or in ready-to-use dispensing systems. By way of example, the packaging may take the form of an aerosol (e.g., in conventional aerosol containers), in liquid form in a pump equipped container (e.g., such as a nasal spray bottle or pump spray bottle), or a squeeze bottle. Typically, the dispensing system may include a pump mechanism to build the necessary pressure to produce an aerosol form for individual consumption.

In another embodiment, the nanoemulsion including the antigen and/or antimicrobial actives may be dried and delivered as such. For example, a dried nanoemulsion delivered orally or into the mucosal membrane may be rehydrated by moisture present so that upon contact with moisture the actives are released.

The nanoemulsion composition may be provided for large-scale use, or for smaller-scale individual use on an as desired basis. In any case, the emulsion composition has wide ranging applicability, and can be used in a broad array of vaccine applications and oral care sprays or rinses while being safe, stable for long periods of time, and cost efficient. Advantageously, the employed hydrophobic flavorants are protected from oxidation, preventing degradation of the flavorant and resulting generation of malodorous products.

Additional details of suitable nanoemulsions may be found in the inventors prior U.S. Publication No. 2009/0196972, herein incorporated by reference in its entirety.

### XI. Exemplary Formulations

A nanoemulsion prepared with an essential oil, TWEEN, propylene glycol, cationic guar gum (e.g., JAGUAR) and water results in an exothermic reaction upon dissolution. A set of experiments was conducted to determine which ingredients are responsible for the exothermic reaction. First, a solution of propylene glycol was diluted with water yielding about a 5° F. to 7° F. temperature change. Second, separate solutions of TWEEN 80 and TWEEN 20 were diluted with water resulting in about a 10° F. to 15° F. temperature change. Finally, a concentrated nanoemulsion composition of about 57% Tween 20, 11% essential oil, 16% propylene glycol and 16% water was diluted with water which resulted in about a 15° F. to 20° F. temperature increase. These results indicate that both the propylene glycol and the TWEEN surfactants are responsible for the exothermic reaction that results when the concentrated composition is diluted with water.

The enthalpy change of solution refers to the quantity of heat produced or absorbed when one mole of a substance is dissolved in a large volume of a solvent at constant pressure. In strong dipole interactions, the solute-solvent interaction may exceed the solvent-solvent interaction, resulting in excess energy in the form of heat, in which case the dissolution process is an exothermic reaction with a negative heat of solution. The heat of solution of a substance is defined in similar terms—by energy absorbed, or endothermic energy, and energy released, or exothermic energy (expressed in “negative” kJ/mol). Heat of solution is an important factor in solubility analysis because a large negative heat of solution associated with exothermic reactions correlates to increased solubility.

Generation of heat as the adjuvant nanoemulsion compositions are diluted with water is beneficial for a number of reasons. First, the generation of heat causes more of the essential oil to be dispersed into the air, which enhances the fragrance and taste of the product to the consumer. Therefore, less flavorant (e.g., an essential oil) may be used in the food grade emulsion while maintaining the same enhanced activity and taste benefit to a consumer. Second, the increase in heat upon dilution of the concentrated nanoemulsion may increase the solubility of the active pharmaceuticals (e.g., antigen or anti-microbial agent) within the heated nanoemulsion.

Exemplary concentrated food grade emulsion compositions are provided within Table 1 below:

TABLE 1
Concentrated Nanoemulsion Formulations

| | Example | Example | Example | Example |
|---|---|---|---|---|
| Component | 1 | 2 | 3 | 4 |
| Antigen SEB Toxoid F | 0.06% | 0.0% | 0.0% | 0.0% |
| Extrinsic Nanoemulsion | | | | |
| Antigen SEB Toxoid F | 0.0% | 0.06% | 0.0% | 0.0% |
| Intrinsic Nanoemulsion | | | | |
| Antigen SEB Toxoid F | 0.0% | 0.0% | 0.05% | 0.0% |
| Conjugated with Proteosomes | | | | |
| Antigen SEB Toxoid F | 0.0% | 0.0% | 0.0% | 0.05% |
| Extrinsic Nanoemulsion | | | | |
| TWEEN 20 | 4.0% | 10.0% | 20.0% | 40.0% |
| Food Grade Propylene Glycol | 2.0% | 5.0% | 10.0% | 15.0% |
| Peppermint Essential Oil | 2.0% | 5.0% | 10.0% | 15.0% |
| Cationic Guar | 2.0% | 5.0% | 0.0% | 0.0% |
| Water | Balance | Balance | Balance | Balance |

TABLE 2
Concentrated Nanoemulsion Formulations

| Component | Example 5 | Example 6 | Example 7 | Example 8 |
|---|---|---|---|---|
| Antigen SEB | 0.0% | 0.05% | 0.0% | 0.0% |
| Toxoid C | | | | |
| Antigen SEB- | 0.05% | 0.0% | 0.0% | 0.05% |
| Toxoid F | | | | |
| Antigen SEB- | 0.0% | 0.0% | 0.05% | 0.0% |
| Toxoid C | | | | |
| Complexed to | | | | |
| proteosomes | | | | |
| TWEEN 20 | 4.0% | 10.0% | 20.0% | 40.0% |
| Sodium Lactate | 1.0% | 0.0% | 5.0% | 0.0% |
| Sodium | 2.0% | 0.0% | 0.0% | 0.0% |
| Gluconate | | | | |
| Glycerin | 0.0% | 5.0% | 0.0% | 10.0% |
| Peppermint | 2.0% | 5.0% | 10.0% | 20.0% |
| Essential Oil | | | | |
| Cationic Guar | 2.0% | 0.0% | 10.0% | 0.0% |
| Water | Balance | Balance | Balance | Balance |

### XII. Experimental

Aspects of the inventive compositions are illustrated by the specific formulations described below without being limited to those formulations. The results show that each of five testers found the taste of a regular (not characterized by nano-sized droplets) essential oil emulsion at 0.004% to be weaker than that of the nanoemulsion composition with the same amount of essential oil. Furthermore four of the testers found that the standard essential oil emulsion at 0.008% was also weaker than the nanoemulsion composition with half of the amount of essential oil, i.e., 0.004%. In addition, three of the testers found that the standard composition with 0.025% essential oil was comparable to the nanoemulsion composition at 0.004%. This taste test shows that the nanoemulsion composition at 0.004% oregano essential oil appears stronger than standard essential oil emulsions with the same amount of essential oil and even appears stronger than essential oil compositions with two or more times the amount of essential oil.

Table 3 summarizes the findings of the testing in which + means the sample was stronger, − means the sample was weaker, and = means the sample was equal as compared to the standard.

TABLE 3
Taste Test Oregano in water (Dilution of 0.004% nanoemulsion)

| Sample | Garlic Essential | Tester | Tester | Tester | Tester | Tester |
|---|---|---|---|---|---|---|
| No. | Oil+ | 1 | 2 | 3 | 4 | 5 |
| 1a | Oregano Oil | stan- | stan- | stan- | stan- | stan- |
| | Nanoemulsion | dard | dard | dard | dard | dard |
| | 0.004% | | | | | |
| | (tasting standard) | | | | | |
| 2a | Oregano Oil | − | − | − | − | − |
| | 0.004% | | | | | |
| 3a | Oregano Oil | − | − | = | − | − |
| | 0.008% | | | | | |
| 4a | Oregano Oil | + | = | + | = | = |
| | 0.025% | | | | | |
| 5a | Oregano Oil | = | = | = | = | = |
| | Nanoemulsion | | | | | |
| | 0.004% | | | | | |

Table 4 is similar to Table 3 in that it uses the same format for testing similar samples of garlic essential oil formulations against a comparable nanoemulsion of garlic oil in water. The same plus, minus and equal signs are used for reporting the testing results of the tasters.

The results show that each of the five testers found the taste of the regular essential oil composition at 0.005% and at 0.009% were both weaker than that of the nanoemulsion composition with the same amount of garlic essential oil. Furthermore two of the testers found that the standard garlic essential oil at 0.015% was comparable to the nanoemulsion composition with about one third the amount of garlic essential oil, 0.005%. The other three testers found it to be weaker. This taste test shows that the nanoemulsion composition at 0.005% garlic essential oil appears stronger than similar standard essential oil compositions with the same amount of essential oil and even appears stronger than essential oil compositions with two or more times (e.g., 3 times) the amount of essential oil.

TABLE 4
Taste Test Garlic in water (Dilution of 0.005% nanoemulsion)

| Sample | Garlic Essential | Tester | Tester | Tester | Tester | Tester |
|---|---|---|---|---|---|---|
| No. | Oil+ | 1 | 2 | 3 | 4 | 5 |
| 1b | Garlic Oil | stan- | stan- | stan- | stan- | stan- |
| | Nanoemulsion | dard | dard | dard | dard | dard |
| | 0.005% | | | | | |
| | (tasting standard) | | | | | |
| 2b | Garlic Oil | − | − | − | − | − |
| | 0.005% | | | | | |
| 3b | Garlic Oil | − | − | − | − | − |
| | 0.009% | | | | | |
| 4b | Garlic Oil | − | = | = | − | − |
| | 0.015% | | | | | |
| 5b | Oregano Oil | = | = | + | = | + |
| | Nanoemulsion | | | | | |
| | 0.005% | | | | | |

The taste test results show that the tested nanoemulsion formulations are comparable to standard formulations with at least twice the amount of essential oil flavorants. In addition, the nanoemulsions with encapsulated flavorant essential oils showed better stability and less oxidation in comparison with standard emulsion compositions. The compositions in Tables 3 and 4 were evaluated by a tasting panel after seven months and twelve months of room temperature storage. At seven months each of the standard, non-encapsulated essential oil in water formulations showed typical signs of oxidation and deterioration including rancid aroma and bitter flavor. In comparison, the nanoemulsion encapsulated essential oil in water formulations did not show any of the typical signs of oxidation or deterioration even after twelve months of storage at room temperature. From the stability testing it appears that the nanoemulsion formulations provide better overall stability, preventing undesirable changes within the compositions that result in rancid aroma and bitter flavor.

Additional testing was performed to determine the effect of various components on droplet size. Table 5 records the effect of the surfactant (TWEEN 20).

| Sample No. | 5A | 5B | 5C |
|---|---|---|---|
| Oil | 1% | 1% | 1% |
| | Garlic Oil | Garlic Oil | Garlic Oil |
| TWEEN 20 | 10% | 15% | 20% |
| Ethanol | 2% | 2% | 2% |
| Sorbitol | 30% | 30% | 30% |
| Water | 57% | 52% | 47% |
| Notes | Cloudy | Clear | Clear |
| Mean Diameter | 28.9 nm | 35.7 nm | 43.7 nm |

As noted, sample 5A was cloudy, rather than clear. It was tested for stability at 70° F., and samples 5B and 5C were stable at that temperature.

Tables 6-1 and 6-2 record the effect of the ethanol.

TABLE 6-1
The Effect of Ethanol on Droplet Size

| Sample No | 6A | 6B | 6C | 6D | 6E |
|---|---|---|---|---|---|
| Oil | 1% | 1% | 1% | 1% | 1% |
| | Onion | Onion | Garlic | Garlic | Garlic |
| TWEEN 20 | 22% | 22% | 22% | 22% | 22% |
| Ethanol | 0 | 4 | 0 | 4 | 0 |
| Sorbitol | 30 | 30 | 30 | 30 | 15 |
| Water | 47 | 43 | 47 | 43 | 62 |
| Notes | Clear | Clear | Clear | Clear | Clear |
| Mean Dia. | 34.9 nm | 40.8 nm | 35.2 nm | 42.4 nm | 17 nm |
| Stability 35° F. | | Yes | | Yes | Yes |
| Stability 70° F. | Yes | Yes | Yes | Yes | Yes |
| Stability 100° F. | | Yes | | Yes | Yes |

TABLE 6-2
The Effect of Ethanol on Droplet Size (continued)

| Sample No | 6F | 6G | 6H | 6I | 6J | 6K |
|---|---|---|---|---|---|---|
| Oil | 1% | 1% | 1% | 1% | 1% | 1% |
| | Garlic | Garlic | Garlic | Garlic | Garlic | Garlic |
| TWEEN 20 | 22% | 22% | 22% | 22% | 22% | 20% |
| Ethanol | 2 | 0 | 1 | 2 | 0 | 2 |
| Sorbitol | 15 | 2 | 2 | 2 | 2 | 2 |
| Water | 61 | 75 | 74 | 73 | 75 | 75 |
| Notes | Clear | Clear | Clear | Clear | Clear | Clear |
| Mean Dia. | 30.5 nm | 10.6 nm | 10.7 nm | 11 nm | 10.6 nm | 11.6 nm |
| Stability | Yes | | | Yes | | no |
| 35° F. | | | | | | |
| Stability | Yes | Yes | Yes | Yes | Yes | Yes |
| 70° F. | | | | | | |
| Stability | Yes | | | Yes | | Yes |
| 100° F. | | | | | | |

As noted, increasing the ethanol seems to lead to increased mean diameter of the droplets. All tested samples showed temperature stability at 35° F., 70° F., 100° F. except for sample 6K, which was unstable at 35° F., but showed stability at higher temperatures (i.e., 70° F. and 100° F.).

Tables 7-1 and 7-2 record the effect of the sorbitol concentration.

TABLE 7-1
The Effect of Sorbitol on Droplet Size

| Sample No. | 7A | 7B | 7C | 7D |
|---|---|---|---|---|
| Oil | 1% | 1% | 1% | 1% |
| | Garlic | Garlic | Garlic | Garlic |
| TWEEN 20 | 20% | 20% | 22% | 22% |
| Ethanol | 2% | 2% | 0% | 0% |
| Sorbitol | 2% | 30% | 2% | 15% |
| Water | 75% | 47% | 75% | 62% |
| Notes | Clear | Clear | Clear | Clear |
| Mean Dia. | 11.6 nm | 43.7 nm | 10.6 nm | 17 nm |
| Stability 35° F. | No | | | Yes |
| Stability 70° F. | Yes | Yes | Yes | Yes |
| Stability 100° F. | Yes | | | yes |

TABLE 7-2
The Effect of Sorbitol on Droplet Size (continued)

| Sample No. | 7E | 7F | 7G | 7H |
|---|---|---|---|---|
| Oil | 1% | 1% | 1% | 1% |
| | Garlic | Onion | Onion | Onion |
| TWEEN 20 | 22% | 22% | 22% | 22% |
| Ethanol | 0% | 0% | 0% | 0% |
| Sorbitol | 30% | 2% | 15% | 30% |
| Water | 47% | 75% | 62% | 47% |
| Notes | Clear | Clear | Clear | Clear |
| Mean Dia. | 35.2 nm | 13.7 nm | 19.6 nm | 34.9 nm |
| Stability 35° F. | | | Yes | |
| Stability 70° F. | Yes | Yes | Yes | Yes |
| Stability 100° F. | | | Yes | |

As noted, increasing the kosmotrope (e.g., sorbitol) strongly increases mean diameter of the droplets. All tested samples showed temperature stability at 35° F., 70° F., 100° F. except for sample 7A, which was unstable at 35° F., but showed stability at higher temperatures (i.e., 70° F. and 100° F.). Temperature stabilities for each of the samples tested within Tables 5-7 were determined using Confocal Raman Spectroscopy.

The nanoemulsion composition may be used in a concentrated nanoemulsion or it can be provided in a diluted form as already added into a product. These various forms and the wide variety of flavor which can be used to create a nanoemulsion composition provide a number of possibilities for compositions. While the present invention has been described with reference to what are presently considered to be the preferred embodiments, it is to be understood that the invention is not limited to these embodiments. To the contrary, the invention is intended to cover various modifications and equivalent arrangements included within the spirit and scope of the appended claims.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

We claim:

1. An adjuvant vaccine emulsion composition comprising:
(a) an antigen derived from bacteria, fungi, viruses, or parasites;
(b) a food safe nonionic surfactant;
(c) a hydrophobic food flavorant selected from the group consisting of: essential oil, oleoresin, oil-based natural flavors and combinations thereof;
(d) a kosmotrope; and
(e) water;
(f) wherein the composition does not contain emulsifiers, phospholipids, triglycerides, vegetable oils, cholesterol, fatty acids, block polymers, trehalose dimycolate, cell wall skeleton, or mercury preservatives.

2. The adjuvant emulsion composition of claim 1, wherein the composition comprises a polymer.

3. The adjuvant emulsion composition of claim 2, wherein the polymer comprises a single cationic layer.

4. The adjuvant emulsion composition of claim 2, wherein the polymer is a bilayer, wherein a first layer of the bilayer is an anionic polymer and a second layer of the bilayer is a cationic polymer.

5. The adjuvant emulsion composition of claim 1, wherein the kosmotrope comprises a food grade sugar alcohol and wherein said food grade sugar alcohol is selected from the group consisting of: sorbitol, propanediol, xylitol, arabitol, lactitol, maltitol, glycerol, mannitol, isomalt, erythritol, and mixtures thereof.

6. The adjuvant emulsion composition of claim 1, wherein the kosmostrope is selected from the group consisting of: lactate, gluconate, sulfate, adipate, citrate, chloride, carbonate, bicarbonate, phosphate, pyrophosphate, nitrate, acetate and mixtures thereof.

7. The adjuvant emulsion composition of claim 6, wherein the kosmotrope is selected from the group consisting of lactate and gluconate.

8. The adjuvant emulsion composition of claim 7, wherein the kosmotrope is a gluconate.

9. The adjuvant emulsion composition of claim 1, wherein the composition has an average droplet size of about 25 nm or less.

10. The adjuvant emulsion composition of claim 9, wherein the composition is a nanoemulsion which has an average droplet size of about 5 nm to 15 nm.

11. An adjuvant vaccine emulsion composition comprising:
a. an antigen derived from bacteria, viruses or parasites;
b. a food safe nonionic surfactant;
c. a hydrophobic natural flavorant selected from the group consisting of:
essential oil, oleoresin, oil-based natural flavors and combinations thereof;
d. a kosmotrope selected from the group consisting of lactate, gluconate, sulfate, adipate, citrate, chloride, carbonate, bicarbonate, phosphate, pyrophosphate, nitrate, acetate and mixtures thereof; and
e. water; and
f. wherein the composition does not contain emulsifiers, phospholipids, triglycerides, vegetable oils, cholesterol, fatty acids, block polymers, trehalose dimycolate, cell wall skeleton, or mercury preservatives.

12. The adjuvant emulsion composition of claim 11, wherein the composition comprises a bilayer polymer, wherein a first layer of the bilayer polymer is an anionic polymer and a second layer of the bilayer polymer is a cationic polymer.

13. The adjuvant emulsion composition of claim 11, wherein the composition does not contain any additional preservatives, antioxidants or flavorants other than the hydrophobic food flavorant.

14. The adjuvant emulsion composition of claim 11, wherein the composition is dried and can also be rehydrated.

15. The adjuvant emulsion composition of claim 11, wherein the surfactant comprises a sorbitan ester.

16. The adjuvant emulsion composition of claim 11, wherein the emulsion composition does not contain any alcohol.

17. The adjuvant emulsion composition of claim 11, wherein the kosmotrope is a gluconate.

18. An adjuvant vaccine composition in the form of a nanoemulsion consisting essentially of:
a. an antigen derived from bacteria, viruses or parasites;
b. a food safe nonionic surfactant;
c. a hydrophobic food flavorant selected from the group consisting of:
essential oil, oleoresin, oil-based natural flavors and mixtures thereof;
d. a kosmotrope;
e. water; and
f. optionally, osmotic agents, pressure regulators, glycerol, mannitol, preservatives, thiomersal, chlorobutanol, hypochlorous acid, parabens, antioxidants, alpha tocopherol, bioadhesive agents, natural gums, karaya, PGA, pectin, polyacrylic acid, polymethacrylates PAA copolymers, carbopol, carbomer, CMC, PVA and mixtures thereof; and
g. wherein the composition does not contain emulsifiers, phospholipids, triglycerides, vegetable oils, cholesterol, fatty acids, block polymers, trehalose dimycolate, cell wall skeleton, or mercury preservatives.

19. The adjuvant emulsion composition of claim 18, wherein the nonionic surfactant is a sorbitan ester.

20. The adjuvant emulsion composition of claim 18, wherein the kosmotrope is selected from the group consisting of: lactate, gluconate, sulfate, adipate, citrate, chloride, carbonate, bicarbonate, phosphate, pyrophosphate, nitrate, acetate and mixtures thereof.
